(19) United States
(12) Patent Application Publication
(10) Pub. No.: US 2023/0356144 A1
(43) Pub. Date: Nov. 9, 2023

(54) PROCESS

(71) Applicant: CEMVITA FACTORY, INC., Houston, TX (US)

(72) Inventors: Roger Allen HARRIS, Houston, TX (US); Rajesh GUPTA, Houston, TX (US); Kevin Patrick KINCAID, Houston, TX (US); Tahereh KARIMI, Houston, TX (US); Matthew SHAPIRO, Houston, TX (US); Nymul KHAN, Houston, TX (US); Marcio Luis Busi DA SILVA, Houston, TX (US); Edris TAHER, Houston, TX (US)

(21) Appl. No.: 18/312,893

(22) Filed: May 5, 2023

Related U.S. Application Data
(60) Provisional application No. 63/364,275, filed on May 6, 2022.

(51) Int. Cl. B01D 53/18; B01D 53/14; C12N 1/20; C12M 1/00; C12M 1/34; C12N 1/32

(52) U.S. Cl. CPC B01D 53/1475; B01D 53/1412; B01D 53/1425; B01D 53/18; C12M 23/58; C12M 29/08; C12M 41/12; C12M 41/26; C12M 41/34; C12N 1/20; C12N 1/32; B01D 2252/60

(57) ABSTRACT

The present application provides a process for sequestering carbon dioxide to produce a biomass containing reaction product, the process comprising the steps of:
I. contacting a raw carbon dioxide-containing feedstock with an absorption or dissolution medium to form a reagent stream comprising dissolved or absorbed inorganic carbon at least in the form of HCO₃⁻ and CO₃²⁻ wherein the HCO₃⁻ :CO₃²⁻ molar ratio in the reagent stream is at least about 0.8;
II. contacting at least a portion of the reagent stream with a microbial broth in a bioreactor to produce a biomass-containing reaction product;
III. separating the biomass-containing reaction product into a biomass product and a liquid stream; and
IV. recycling at least a portion of the liquid stream to step i. of the process for use as, or as part of, the absorption or dissolution medium,
wherein the pH of the absorption or dissolution medium is controlled to maintain the HCO₃⁻ :CO₃²⁻ molar ratio in the reagent stream at least about 0.8.

### FIELD

This application claims priority to U.S. Provisional Pat. Application No.63/364,275, filed May 6, 2022. The entirety of the foregoing mentioned application is herein incorporated by reference.

The present application relates to a process for sequestering carbon dioxide to produce a biomass-containing reaction product.

### BACKGROUND

US2014295531 describes a method for the capture and conversion of CO₂ from a gaseous stream for the cultivation of algae. The disclosed method includes contacting a gaseous stream comprising CO₂, with a first absorbent liquid stream comprising an enzyme capable of converting liquid-absorbed CO₂ into a more liquid-soluble inorganic carbon, allowing the CO₂ to be absorbed by said first absorbent liquid and allowing the liquid-absorbed CO₂ to be converted into said more soluble inorganic carbon, separating the first liquid stream comprising both the enzyme and the dissolved inorganic carbon into a second and a third liquid stream, wherein said second liquid stream comprises, relative to said third liquid stream, a higher concentration of the enzyme, recycling said enzyme by supplying the enzyme in said second liquid stream back, together with a portion of the absorbent liquid, to be contacted with the gaseous stream, contacting said third liquid stream with a microorganism capable of converting liquid-solubilised inorganic carbon into oxygen and/or biomass, allowing the conversion of the liquid-soluble inorganic carbon by said microorganism, thereby regenerating the absorbent liquid, and recycling the regenerated absorbent liquid to be contacted with the gaseous stream.

WO2013022349 describes another method for advantageously combining the reduction of CO₂ emission with algal growth. The disclosed method includes contacting a gas comprising carbon dioxide with an absorbent liquid, allowing the absorbent liquid to absorb carbon dioxide from the gas, contacting the absorbent liquid comprising the absorbed carbon dioxide to with an algal culture, allowing the algal culture to convert the carbon dioxide from the absorbent liquid, thereby regenerating the absorbent liquid and promoting the growth of algae in said algal culture.

US2013319059 describes an integrated method culturing algae or cyanobacteria, comprising the steps of i) capturing CO₂ from a source of CO₂ through a solvent; ii) converting captured CO₂ into bicarbonate; iii) culturing alkaliphilic algae or alkaliphilic cyanobacteria using said bicarbonate as a carbon source to produce algal bioproducts; iv) using spent medium from said step of culturing as regenerated solvent to absorb CO₂ in said step of capturing; and v) repeating steps i) to iv). This disclosure describes the use of alkaline conditions to capture CO₂ into bicarbonate, but the disclosed method is mediated by photosynthetic bioprocesses (algae and cyanobacteria) and is apparently unconcerned with the challenges of manipulating the chemical environment of industrial processes for efficacious conversion of carbon dioxide to biomass.

Similarly, EP3284827A1 discloses the use of an enzyme (carbonic anhydrase) to convert CO₂ into bicarbonate and again is essentially concerned with the cultivation of algae by a bio-photosynthetic process and pays no or little attention to the demands of an effective industrial process for carbon dioxide-to-biomass conversion.

The same goes for WO2013106932A1 which concerns the use of biocatalysts in an enzymatic process for capturing CO₂ and converting it to bicarbonate for algal production.

However, none of these published documents mentions satisfactory means for controlling the pH of an industrial process for CO₂-to-biomass conversion. Further, none of the publication recognise the importance of such pH control for selecting the chemical conditions conducive to the conversion, especially as concerns the HCO₃⁻:CO₃²⁻ molar ratio in the raw material, nor the effective balancing of those conditions by means of a recycle stream in an industrial process.

The present application recognises that in a process for sequestering carbon dioxide to produce a biomass-containing reaction product, compositional pH and its attendant impact and influence from recyclate is a key parameter which impacts the bicarbonate concentration (relative to carbonate concentration), the main carbon source for biomass production by microbes. The state-of-the-art contains no satisfactory teaching or indication as to how to control and maintain an efficacious pH environment in such a process in order to optimise carbon capture and microbial growth.

### SUMMARY

One of the objects of the present application is to provide an improved process for sequestering carbon dioxide to produce a biomass-containing reaction product, in which the pH in the system is controlled at least partly by means of a recycle stream to engineer chemical conditions conducive to CO₂-to-biomass conversion, and to enhance the productivity of such conversion.

Another object of the present application is to provide an optimised process that is energy efficient and economical when compared to conventional methods.

### BRIEF DESCRIPTION OF DRAWINGS

FIG. 1 depicts a schematic diagram of a process for sequestering carbon according to the present application.

FIG. 2 graphically represents total solids content (TSS) and pH of the reactor media pursuant to Example 2.

FIG. 3 graphically represents speciation of inorganic carbon as a function of pH pursuant to Example 2.

### DETAILED DESCRIBTION

The present application provides an improved process for sequestering carbon dioxide to produce a biomass-containing reaction product, in which the pH in the system is controlled at least partly by means of a recycle stream to engineer chemical conditions conducive to CO₂-to-biomass conversion, and to enhance the productivity of such conversion.

The application further provides an optimised process that is energy efficient and economical when compared to conventional methods.

According to a first aspect of the present application, there is provided a process for sequestering carbon dioxide to produce a biomass-containing reaction product, the process comprising the steps of:
I. contacting a raw carbon dioxide-containing feedstock with an absorption or dissolution medium to form a reagent stream comprising dissolved or absorbed inorganic carbon at least in the form of HCO₃⁻ and CO₃²⁻ wherein the HCO₃⁻ :CO₃²⁻ molar ratio in the reagent stream is at least about 0.8;
II. contacting at least a portion of the reagent stream with a microbial broth in a bioreactor to produce a biomass-containing reaction product;
III. separating the biomass-containing reaction product into a biomass product and a liquid stream; and
IV. recycling at least a portion of the liquid stream to step i. of the process for use as, or as part of, the absorption or dissolution medium,
wherein the pH of the absorption or dissolution medium is controlled to maintain the HCO₃⁻:CO₃²⁻ molar ratio in the reagent stream at least at about 0.8.

Preferably the HCO₃⁻:CO₃²⁻ molar ratio in the reagent stream is at least about 0.9, and the pH of the absorption or dissolution medium is controlled to maintain the HCO₃⁻:CO₃²⁻molar ratio in the reagent stream at least at about 0.9.

The pH of the absorption or dissolution medium is preferably controlled to facilitate its capacity to absorb or dissolve carbon dioxide and to contribute to reaction conditions in the bioreactor conducive to production of the biomass-containing reaction product.

The required pH control may be effected by biological means (e.g. selection of suitable microbes and/or microbial digestion conditions), chemical means (e.g. by adjusting the pH of the absorption or dissolution medium or of the recycle stream which contributes to it), or by both. Typically, the pH of the absorption or dissolution medium is at least partly controlled by adjusting the pH of the recycling liquid stream, optionally before the stream is supplied to step i.

The pH of the absorption or dissolution medium may for example be adjusted by the addition of an aqueous make-up stream. Often it will be desirable to increase the pH of the recycle stream within a desired range, which can be achieved by the addition of alkaline aqueous make-up stream.

The process of the application is particularly effective in reducing the energy required for carbon capture and utilisation. In conventional chemical processes a large part of energy required for carbon capture is consumed in regenerating the solvent used for CO₂ absorption. In the present application, biological systems are used to consume the dissolved CO₂ present in the form of bicarbonate in solution, thereby producing the recyclable solvent and saving the energy conventionally needed for regeneration of solvent.

The source of the fluid feedstock stream may for example be flue gas, ambient air, and/or any other process gas and/or liquid stream containing carbon dioxide. The use of such sources advantageously aids in reducing otherwise detrimental carbon dioxide emissions.

The contacting of the raw carbon dioxide-containing feedstock with an absorption or dissolution medium to form a reagent stream comprising dissolved inorganic carbon may be affected in a suitable vessel such as a gas contactor or absorption column. The absorption/dissolution medium in the recycle stream typically has a relatively low carbon dioxide content, which is substantially increased by contact with the raw carbon dioxide-containing feedstock. Thus, the process may further comprise increasing the carbon content of the recycle stream by dissolving or absorbing CO₂ into the recycle stream, for example with the aid of a gas contactor or absorption column.

There are various types of commercially available gas contactor or absorption columns known in the art. The gas contactor or absorption column suitable for use in the present application may be dependent on the composition of the inlet fluid stream. Known commercially available contactors my include, but are not limited to, absorption columns with unstructured packing, absorption columns with structured packing or absorption columns with trays.

The packing of the gas contactor or absorption columns or trays utilised are specifically designs to improve gas and/or liquid mass transfer. Such designs are known and can be found, for example from Perry’s Chemical Engineers’ Handbook. New York: McGraw-Hill, 1984.

The optimal column design (height, width, packing) upstream to the bioreactor suitable for the present application can be determined by simulating a CO₂ absorber using commercially available modelling tools, from example Aspen plus.

The raw carbon dioxide-containing feedstock may be pre-supplied to such a gas contactor or absorption column before entering the bioreactor. The gas contactor or absorption column and the bioreactor may therefore be provided as separate zones. Alternatively, the gas contactor or absorption column and bioreactor may be provided as a single hybrid zone. In this case, it will be understood that in the process of the present application the gas contactor or absorption column and the bioreactor may be a single vessel, for example a tubular plug flow reactor. In this case, the recycle stream would be fed directly into the bioreactor in addition to the fluid stream comprising carbon dioxide.

The raw carbon dioxide-containing feedstock may be treated within the gas contactor or absorption column to produce a carbon-rich feedstock liquid (the reagent stream comprising dissolved or absorbed inorganic carbon) and an off-gas substantially free of carbon dioxide. For example, the gas contactor or absorption column may be configured to absorb carbon dioxide from the raw feedstock (for example, ambient air, flue gas or any other process gas stream containing CO₂) to produce a concentrated carbon liquid stream (as reagent stream) and an off-gas substantially free of carbon dioxide.

The off-gas substantially free from carbon dioxide may be utilised in any commonly known process. For example, the off-gas free from carbon dioxide may be used in processes such as hot air regeneration of adsorbents or used as a compressed air feed.

The raw feedstock may optionally be fed through a pressure booster prior to entering the gas contactor or absorption column and/or the bioreactor. Optionally the raw feedstock may be pre-treated prior to entering the process, for example by clean-up or concentration.

The raw feedstock may comprise up to about 100% v/v carbon dioxide, but may comprise various other materials, such as are typically found in industrial effluents, flue gases, off-gases and/or ambient air.

The amount of carbon dioxide may be balanced mainly by nitrogen, water and oxygen. Additional trace amounts of SOx, NOx, H₂S, particulate matter, CO and other compounds typically present in industrial gas, petrochemical, or other heavy industrial operations may be present.

The reagent stream comprises HCO₃⁻ and CO₃²⁻ and may also include other forms of dissolved or absorbed inorganic carbon (such as carbon oxides and other carbonic compounds - carbon monoxide; carbon dioxide; cyanide; cyanate; thiocyanate; and/or carbides, for example). So far as the HCO₃⁻ and CO₃²⁻ content of the reagent stream is concerned, the molar ratio of HCO₃⁻:CO₃²⁻ is at least about 0.8, preferably at least about 0.9 and may for example be from about 0.8 to about 1.5, for example from about 0.9 and to about 1.5.

The reagent stream may have a pH of from about 8 to about 11, for example from about 8.5 to about 11, from about 9 to about 11, for example from about 9.5 to about 10.5. A pH of between about 8 or about 9 and about 11 ensures that there is enough bicarbonate in the process to feed microbes in the bioreactor, thereby facilitating the production of biomass (or any other final product for which the biological system using the process is designed).

It is known in the art that bicarbonate manifests in aqueous solution at a pH of between about 6 and 10.5, preferably between 9 and 9.5, for optimal conditions for bicarbonate. By controlling the pH of the reagent stream as stipulated above, the process of the application provides optimal conditions for ensuring maximum carbon dioxide adsorption whilst still maintaining a sufficient bicarbonate molar ratio to provide maximal microbe growth.

It is desirable to maintain the pH of the bioreactor to optimise microbial growth. In certain embodiments, the microbial broth in the bioreactor may have a pH of greater than about 8, or greater than about 9, or greater than or equal to about 10. In one embodiment, the microbial broth in the bioreactor has a pH of greater than or equal to about 10.

Controlling the microbial broth to be at about pH 10 or above enables a higher carbon dioxide absorption efficiency of the recycle stream (i.e. a carbon lean solvent). Advantageously, a high volume of recycle stream will reduce the amount of make-up water and optionally, sodium hydroxide, that needs to be added into the system.

Suitable microbes for use in the present application may be bicarbonate metabolising and/or pH maintaining.

Preferably the microbial broth used in the process of the application comprises at least one non-algal and/or non-photosynthetic microorganism. More preferably, the microbial broth contains algae and/or photosynthetic microorganism, if at all, in an amount of less than 50 wt%, less than 25 wt %, less than 10 wt %, less than 5 wt % or less than 1 wt % of all microbiological materials present in the broth.

During the conversion of bicarbonate to biomass or any other product by microbes, the pH of the microbial broth in the bioreactor is likely to increase, thereby reducing the overall efficiency of the process and reducing microbial growth. Therefore, in certain embodiments the pH of the microbial broth in the bioreactor may be controlled by the injection of a gas comprising carbon dioxide.

The carbon dioxide may be sourced from waste or a by-product of a downstream process. The downstream process may include conversion of produced biomass or product in bioreactor to biofuels or any other chemicals.

The inventors have advantageously found that using carbon dioxide produced in a downstream process facilitates close control of the chemical and pH conditions of the microbial broth in the bioreactor whilst removing carbon from the environment following its production elsewhere.

The gas comprising carbon dioxide injected into the bioreactor may have a concentration of CO₂ of greater than 50% v/v, or greater than 75% v/v, or greater than 90% CO₂ v/v.

The microbial broth comprises a microbial population in combination with a carrier medium which may comprise an aqueous alkaline solution (alkalinity being provided by, for example, sodium hydroxide or potassium hydroxide). The aqueous alkaline solution may comprise waste water from an industrial process. The carrier medium may alternatively or as well comprise alternative alkaline material such as ammonia, one or more amines, and may also include one or more enzymes (carbonic anhydrase for example).

The inventors have found that such an environment in the bioreactor provides a controlled environment that achieves optimal microbial growth.

The microbial broth efficiency of carbon dioxide removal is at least 50%, or at least 60%, or at least 70%.

Examples of microbiological materials suitable for use in the microbial broth of present application include, but are not limited to:
Photoautotrophs (e.g. algae (e.g. Laminaria, Undaria, Gracilaria, Ascophyllum, Euchetuna, Macrocystis, Lessonia, Chondrus, Sargassum, Hizikia)),
Cyanobacteria (e.g. Chamaesiphon, Chroococcidiopsis, Arthrospira, Anabaena, Chlorogloeopsis, Chroococcus, Dermocarpella, Geitlerinema, Anabaenopsis, Fischerella Cyanothece, Myxosarcina Leptolyngbya Aphanizomenon, Dactylococcopsis, Pleurocapsa, Lyngbya, Calothrix, Gloeobacter, Stanieria, Microcoleus, Cylindrospermum, Gloeocapsa, Xenococcus, Oscillatoria, Microchaete, Gloeothece, Pseudanabaena, Nodularia, Microcystis, Spiiulina, Nostoc, Synechococcus, Symploca, Scytonema, Synechocystis Tolypothrix),
Non-photosynthetic or non-photoautotrophs such as methanogenic consortia (e.g. Cupriavidus necator, Clostridium thermocellum, Clostridium ljundahlii and other microbes from the Clostridium genus.),
Auto-chemo-lithotropics. (Examples of bacteria include those of the genera Acidithiobacillus (e.g. Acidithiobacillus ferrooxidans), Acidophilus (e.g. Acidophilus thioosidans, Acidophilus caldus), Leptospirillum (e.g. Leptospirillum ferrooxidans), Thiobacillus (e.g. Thiobacillus ferrooxidans, Thiobacillus thiooxidans, Thiobacillus organoparus), Thermothrix (e.g. Thermothrix thiopara) and / or Sulfobacillus. Examples of archaea include those of the genera Pyrococcus (e.g. Pyrococcus furiosus), Pyrobaculum (e.g. Pyrobaculum islandicum), Sulfolobus (e.g. Sulfolobus acidocaldarius), Acidianus, Ferroplasma, Metallosphaera, and / or Thermoplasma.)
Alkaliphiles, either autotrophic, heterotrophic, and/or mixotrophic (examples include microbes from the genera Microbacterium, Cellulosimicrobium, Kocuria, Halorhodospira, Natronomonas, Thiohalospira, Thiomicrospira (e.g. Thiomicrospira cyclica), Thioalbus, Thioalkalivibrio, Nocardiopsis, Paenibacillus, and Stenotrophomonas).
Thermophiles (examples include microbes from the genera Streptomyces Acidithiomicrobium, Caldicellulosiruptor, Sulfolobus, Thermotoga, Thermococcus, Pyrococcus, Metallosphaera, Thermoanaerobacter, and Thermotoga),
Engineered microbes for example, any of the microbes mentioned herein which are genetically manipulated to overexpress native genes, under-express or delete native genes, or introduce non-native genes,
Bacterial consortia (For example, at least two or more of the organisms mentioned herein), alternatively, rationally designed consortium may be used to enable pH maintenance / bicarbonate metabolism as well as the production of compounds of commercial interest,
Enzymes and/or proteins, for example, any protein and/or enzyme, or combination of proteins or enzymes isolated from the microbes mentioned herein.

The microbiological materials used in the present application may be dependent on the final product or the biomass.

In certain embodiments, the microbiological materials comprise at least one alkaliphile. Advantageously, alkaliphiles operate at the pH greater than 10, thereby enhancing the absorption efficiency of the recycled solvent. Additionally, alkaliphiles reduce the solvent’s degradation by using a low temperature regeneration process, thus reducing the solvent make-up. The use of alkaliphiles further provides several other benefits to the optimisation and environmental impact of the process according to the present application including eliminating heat required to regenerate the solvent by using microbial process; improving the bioreactor productivity by keeping carbon dioxide in the HCO₃⁻ form in liquid through pH control of concentrated carbon liquid stream coming from the gas contactor or absorption column; reducing mass transfer limitation by keeping the carbon dioxide in liquid phase; using natural sunlight or artificial light indirectly powered through renewable solar energy for cultivation of autotrophs.

In certain embodiments, the microbiological materials comprise at least one thermophile. Advantageously, thermophiles permit operation of the bioreactor at a higher temperature, thereby improving the efficiency of the bioreactor and reducing the cooling of upstream equipment.

In certain embodiments, the microbiological materials comprise engineered microbes, thereby enhancing bicarbonate metabolism, pH maintenance capability, alkaliphilism, and/or thermophilism.

In certain embodiments, the microbiological materials comprise bacterial consortia, comprising at least two or more of the organisms discussed herein. Alternatively, rationally designed consortium to enable pH maintenance and/or bicarbonate metabolism as well as the production of compounds of commercial interest, may be used.

The biomass containing reaction product may be fed into a biomass separator.

There are various types of commercially available biomass separators known in the art. Known commercially available contactors may include, but are not limited to, a settling tank, a flocculation/coagulation process, dissolved air floatation, electrocoagulation, filtration (crossflow, pressure, vacuum, press, depth), cyclones, centrifuges, or any combination thereof.

It has been found that the process according to the present application advantageously maximizes both biomass recovery and volume of high pH liquid to be recycled (recycle stream).

The biomass-containing reaction product comprises at least biomass. The biomass-containing reaction product may comprise at least biomass and a liquid stream. The biomass-containing product may for example comprise a biomass, at least one other reaction product and at least one liquid stream. The liquid stream may comprise water blow down to be disposed of and a liquid stream to be recycled back into the process. The liquid stream to be recycled may be a carbon lean stream. This stream may be referred to as the “recycle stream”.

It is desirable to control the pH of the recycle stream to control the maintenance of the desired bicarbonate molar ratio throughout the process of the present application to optimise the overall processing conditions.

The present application has illustrated unexpected advantages associated with controlling the pH of the recycle stream. One advantage is that by controlling the pH of the recycle stream, the energy required for carbon capture and utilisation is reduced, compared to conventional methods in the art. A process according to the present application therefore facilitates an energy efficient biological route to carbon dioxide air capture pathway, for example, through the use of only solar power or waste/inexpensive feedstock.

Maximizing the volume of the liquid stream that is recycled will advantageously reduce the amount of make-up water and caustic that needs to be added into the process.

The recycle stream may be a “carbon lean solvent”. By “carbon lean” is preferably mean that the recycle stream comprises carbonaceous compounds, if at all, in an amount of less than about 10% mol %, preferably less than 5 mol%, more preferably less than 2 mol% and most preferably less than about 1 mol%.

The recycle stream may have a pH of between about 10.5 and about 11.75. In one embodiment, the recycle stream has a pH of from about 11 to about 11.6, for example from about 11 to 11.5.

Advantageously, a pH of between about 10.5 and about 11.75 prevents unwanted or undesirable live bacteria (other than those responsible for CO₂ to biomass conversion in the bioreactor) from entering in live form or from thriving in the gas contactor or absorption column.

In some embodiments, make-up water or solvent may be added to the recycle stream prior to the recycle stream returning to the gas contactor or absorption column and/or bioreactor.

If the pH of the recycle stream is not within the desired range reference above, an alkaline compound, for example sodium hydroxide, may be added into make-up water to maintain the pH in the desired range. Therefore, in some embodiments the make-up water comprises such an alkaline compound.

The alkaline compound, for example sodium hydroxide, may be added to the make-up water to increase the pH of the make-up water to from about 12 to about 13. Preferably, the resulting pH of the make-up water is from about 12.25 to about 12.75, for example about 12.5.

The inventors of the present application have found that by increasing pH of the make-up water, optimisation of the overall process is achieved. Firstly, the increase in pH can sterilize the recycle stream. Sterilization of the recycle stream can further be achieved by any methods known in the art, including, but not limited to, raising the pH of the recycle stream to prevent growth, adding biocide, adding bleaching agent(s), UV sterilization, gamma irradiation or increasing temperature.

Additionally, the increase in pH was found to improve carbon dioxide absorption and was found to prevent unwanted microbial growth in the gas contactor or absorption column.

The biomass produced from the process according to the present application may have multiple different uses, dependent on the resulting composition. Biomass may be characterised by the molar ratio of carbohydrate:lipid:protein. Different compositions of biomass will be desired depending on the intended end use. For example, biomass with high lipid content will be desirable for biodiesel production. Conversely, high carbohydrate content is more favourable for production of ethanol or biomass and high protein biomass is suited for the production of protein supplements for agricultural feed or nutraceuticals.

The process according to the present application may optionally further comprise at least one downstream process.

The downstream process may comprise a process that produces a gas comprising carbon dioxide, for example a biofuels production process.

By-products or products from the downstream process may advantageously be recycled and used in the process according to the present application, for example in controlling the pH of the bioreactor, thereby improving the environmental impact of the overall process and reducing the need for the introduction of external means.

Advantageously, the process according to the present application can be used to produce biomass with different characteristics.

The biomass produced from the process according to the present application may be used, but not limited to, use as an energy source, a final product, an intermediate product in a different process, or a carbon storage medium.

Preferably the biomass-containing reaction product is at least partly, preferably wholly, inanimate.

Preferably the microbial broth is at least partly, preferably wholly, non-algal and/or non-photosynthetic in nature.

According to a second aspect of the present application, there is provided an apparatus for capturing carbon in a fluid stream to produce a biomass containing reaction product, the apparatus comprising:
i. a bioreactor for producing a biomass containing reaction product from a fluid stream comprising CO₂;
ii. a biomass separator configured to separate the biomass containing reaction product into biomass and a liquid stream;
iii. means for monitoring and controlling the pH of the liquid stream; and
iv. means for recycling at least part of the separated liquid stream to generate at least part of the fluid stream comprising CO₂.

An apparatus according to the present application may further comprise a gas contactor or absorption column to concentrate the fluid stream with high carbon content.

Preferred embodiments of the application are described below by way of example only with reference to the Figures, wherein:

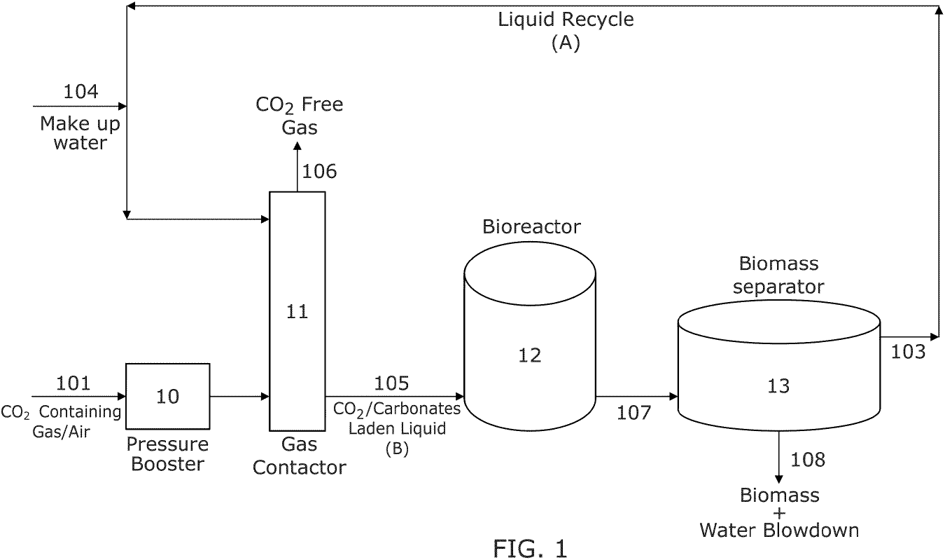

FIG. 1 depicts a schematic diagram of a process for sequestering carbon according to the present application.

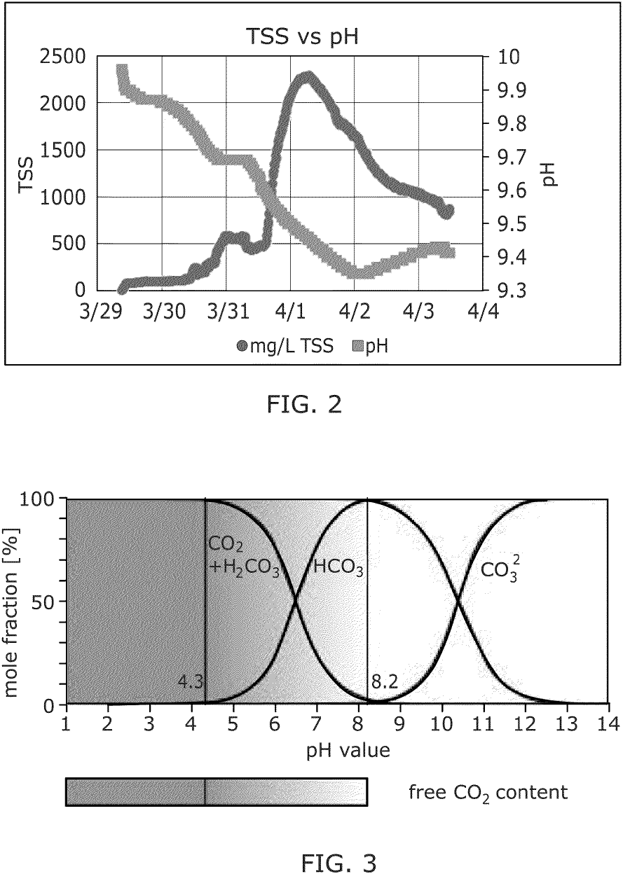

FIG. 2 graphically represents total solids content (TSS) and pH of the reactor media pursuant to Example 2.

FIG. 3 graphically represents speciation of inorganic carbon as a function of pH pursuant to Example 2.

In the process of the present application schematically represented in FIG. 1 a fluid raw material stream comprising carbon dioxide is supplied to the process in line 101.

The flue gas is supplied in line 101 at atmospheric pressure to a pressure booster 10 which increases the pressure of the flue gas to an extent sufficient to overcome the hydraulic head pressure in gas contactor or absorption column 11. Gas contactor or absorption column 11 is supplied in line 103 with a liquid recycle stream from the process, in combination with make-up water from line 104.

In gas contactor or absorption column 11 carbon dioxide in the feedstock stream is dissolved and is supplied on in line 105 to bioreactor 12. A substantially carbon-free off-gas is vented from the gas contactor or absorption column in line 106.

Stream B in line 105 enters bioreactor 12 charged with a microbial broth and maintained under conditions of temperature/pressure/pH effective to ensure consumption by the microbial broth of the stream B to generate a biomass product and a liquid vehicle. Both are supplied on in line 107 to biomass separator 13, from which is recovered in line a biomass product and, in line 108, a recycle stream A which is recycled to gas contactor or absorption column 11 in line 103 after combination with make-up water in line 104.

The following examples are offered by way of illustration of certain embodiments of aspects of the application herein. None of the examples should be considered limiting on the scope of the application.

### EXAMPLES

A plant configured in accordance with FIG. 1 is supplied with fluid raw material supplied in line 101. The fluid raw material in this case is a flue gas having the composition set out in Table 1 (other sources such as power plant or other industrial process could also or alternatively be used):

TABLE 1

Flue gas (fluid raw material) composition

| | |
|---|---|
| Material | %v/v |
| N2 | 75 |
| CO₂ | 10 |
| H2O | 9 |
| O2 | 5 |
| SO2 | 300 (ppm) |
| CO | 700 (ppm) |
| NOX | 150 (ppm) |
| SO3 | 20 (ppm) |

An example composition of the stream arising in FIG. 1, line 105 (stream B) is set out in Table 2:

TABLE 2

feedstock stream - composition

| Material | Concentration (lbmol/hr) |
|---|---|
| CO₂ | 0.47 |
| OH⁻ | 2.89 |
| HCO₃⁻ | 1419.50 |
| CO₃²⁻ | 1043.09 |

An example composition of the substantially carbon-free off-gas (line 106 in FIG. 1) is set out in Table 3:

TABLE 3

carbon-free off-gas - composition

| Material | %v/v |
|---|---|
| N2 | 85 |
| H2O | 9 |
| O2 | 5 |
| Trace components | 1% |

The experiment is repeated with different pH in the make-up water (thereby affecting the pH of the recycle stream A, and consequently the HCO₃⁻: CO₃²⁻ molar ratio in stream B) with the following results and observations.

### Examples 1-6

Six examples of flow compositions according to the present application are set out in Table 4:

TABLE 4

flow composition examples
Examples

| | 1 | 2 | 3 | 4 | 5 | 6 |
|---|---|---|---|---|---|---|
| Recycle Stream (Stream A) | | | | | | |
| Temperature (°F) | 100.20 | 100.20 | 100.20 | 100.20 | 100.20 | 100.20 |
| Pressure (psia) | 15.50 | 15.50 | 15.50 | 15.50 | 15.50 | 15.50 |
| pH | 10.44 | 10.70 | 11.07 | 11.44 | 11.42 | 11.45 |
| CO₂ (lbmol/hr) | 0.01 | 0.00 | 0.00 | 0.00 | 0.00 | 0.00 |
| OH⁻· (lbmol/hr) | 18.94 | 34.08 | 77.56 | 165.72 | 170.75 | 194.87 |
| HCO₃⁻ (lbmol/hr) | 298.74 | 169.55 | 74.82 | 29.80 | 33.18 | 38.81 |
| CO₃²⁻ (lbmol/hr) | 1586.67 | 1643.36 | 1670.10 | 1647.02 | 1643.79 | 2375.42 |
| NaOH added with recycle | 35.00 | 35.00 | 35.00 | 35.00 | 35.00 | 50.00 |
| stream (lbmol/hr) | | | | | | |
| Temperature (°F) | 101.31 | 101.37 | 101.46 | 101.73 | 101.60 | 101.73 |
| Pressure (psia) | 15.00 | 15.00 | 15.00 | 15.00 | 15.00 | 15.00 |
| pH | 9.61 | 9.62 | 9.63 | 9.62 | 9.64 | 9.72 |
| CO₂ (lbmol/hr) | 0.47 | 0.44 | 0.42 | 0.41 | 0.41 | 0.35 |
| OH⁻· (lbmol/hr) | 2.89 | 2.98 | 3.04 | 2.73 | 3.07 | 4.06 |
| HCO₃⁻ (lbmol/hr) | 1419.50 | 1388.75 | 1368.07 | 1341.74 | 1354.92 | 1531.26 |
| CO₃²⁻ (lbmol/hr) | 1043.09 | 1058.09 | 1069.51 | 1081.31 | 1075.54 | 1737.12 |
| HCO₃⁻: CO₃²⁻ | 1.3 | 1.3 | 1.3 | 1.2 | 1.2 | 0.9 |
| Total gas moles IN (lbmol/hr) | 26000 | 26000 | 26000 | 26000 | 26000 | 26000 |
| CO₂ IN (lbmol/hr) | 1040 | 1040 | 1040 | 1040 | 1040 | 1040 |
| CO₂ OUT (lbmol/hr) | 453 | 397 | 338 | 285 | 278 | 173 |
| CO₂ removal efficiency (%) | 56 | 62 | 67 | 73 | 73 | 83 |

As can be seen be comparing Example 1 to Example 5, an increase of the pH of the recycle stream (10.44 and 11.42 respectively) increases the carbon dioxide adsorption efficiency, while still maintaining the desired bicarbonate loading to maintain cell growth in the bioreactor.

With reference to the concentrated carbon liquid stream, there is a need to balance the optimum pH to ensure that there is enough bicarbonate in the process to feed microbes in the bioreactor, but also for optimal carbon dioxide adsorption. As can be seen in Table 4, there is a sharp change in the bicarbonate and carbonate molar ratio when the concentrated carbon liquid stream is changed from a pH of 9.64 (Example 5) to 9.72 (Example 6).

The optimum HCO₃⁻: CO₃²⁻ molar ratio typically manifests between a pH of about 9 and 9.5. However, carbon dioxide adsorption is optimised at a higher pH, as can be seen from Table 4.

The present application is concerned with optimally balancing these somewhat conflicting requirements. This is achieved by adjusting the pH (typically of the recycle stream) to control the HCO₃⁻ and CO₃²⁻ molar ratio in the reagent stream to be at least about 0.8, preferably at least about 0.9.

For the avoidance of doubt, all features relating to process for sequestering carbon dioxide also relate, where appropriate to the apparatus for sequestering carbon dioxide and vice versa.

### Example 7

Microorganisms identified as candidates for the described process are set out in Table 5, each proprietary strain (Isolate ID) having 16S homology with the commercially available strain as indicated:

TABLE 5

exemplary microorganisms

| Isolate ID | 16S ID | % Identity |
|---|---|---|
| AD001 | Thiomicrospira cyclica ALM1 | 99.50 |
| AD002 | Thiomicrospira cyclica ALM1 | 99.28 |
| AD003 | Thiomicrospira cyclica ALM1 | 99.49 |
| AD004 | Thiomicrospira cyclica ALM1 | 99.64 |
| KB001 | Thioalbus sp. strain 2226 | 99.20 |
| KB002 | Thioalkalivibrio sp. ALBR3 | 99.50 |
| KB003 | Thioalkalivibrio sp. ALBR3 | 99.57 |
| KB004 | Thioalkalivibrio sp. ALBR3 | 99.64 |

### Example 8 - Optimal pH

In this example, 1000 liters of exemplificatory feedstock media were prepared according to the concentrations set out in Table 6:

TABLE 6

exemplificatory feedstock media - composition

| | |
|---|---|
| Compound | Concentration (g/L) |
| Sodium carbonate | 20.0 |
| Sodium bicarbonate | 10.0 |
| Sodium chloride | 1.0 |
| Potassium phosphate dibasic | 1.0 |
| Magnesium chloride hexahydrate | 0.2 |
| Sodium thiosulfate pentahydrate | 9.9 |
| Potassium nitrate | 1.0 |
| Ammonium chloride | 0.2 |
| Trace Elements | Trace |

Once prepared, 800 L of the media was charged into a stirred tank bioreactor with micro aeration sparged with air. Inoculant containing a consortia containing one or more of the above exemplary strains was charged to the reactor such that the final optical density of the bioreactor at 600 nm was 0.1. The reactor temperature was maintained at 100° F. (37.78° C.).

During the growth of the microbes, the reactor pH was monitored as was the total suspended solids (TSS). The measurement of TSS can be used as in indirect measurement of microbial productivity. The results are graphically represented in FIG. 2 which shows that the maximum productivity, as measured by TSS, occurs at a pH of ~9.4. According to the graph shown in FIG. 3, this corresponds to a bicarbonate to carbonate (HCO₃⁻: CO₃²⁻) molar ratio of 0.9.

### Example 9 - Recycle Stream pH Control

The plant of FIG. 1 was configured to control the pH of the recycle stream by coupling the pH control with a biomass dewatering step. Effluent from the bioreactor is passed to a filtration unit (not shown). Before filtration, additional CO₂ is injected into the effluent steam. After filtration, pressure is released from the recycle stream, resulting in a slight increase in pH. The resulting stream is passed on in line 103 to gas contactor 11. Modelled results are shown in Table 7 below. The pH of the effluent stream is raised from 9.5 to 9.9 in the process. Further adjustment of recycle stream pH may be effected by the provision of alkaline make-up in line 104.

TABLE 7

modelled data

| Description | Flow (gpm) | TDS (mg/L) | Pressure (psi) | pH |
|---|---|---|---|---|
| Bioreactor Effluent Feed | 14 | 55,912 | 0 | 9.5 |
| Bioreactor Effluent Feed following CO₂ injection | 14 | 55,992 | 453 | 9.5 |
| Filter Retentate Stream | 4.19 | 121,708 | 573.1 | 9.7 |
| Filter Permeate (Recycle) Stream | 9.8 | 27,800 | 0 | 9.9 |

The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the present application, which is defined by the following claims. The aspects and embodiments are intended to cover the components and steps in any sequence, which is effective to meet the objectives there intended, unless the context specifically indicates the contrary.

What is claimed is:

1. A process for sequestering carbon dioxide to produce a biomass containing reaction product, the process comprising the steps of:
contacting a raw carbon dioxide-containing feedstock with an absorption or dissolution medium to form a reagent stream comprising dissolved or absorbed inorganic carbon at least in the form of HCO₃⁻ and CO₃²⁻ wherein the HCO₃⁻:CO₃²⁻ molar ratio in the reagent stream is at least about 0.8;
contacting at least a portion of the reagent stream with a microbial broth in a bioreactor to produce a biomass-containing reaction product;
separating the biomass-containing reaction product into a biomass product and a liquid stream; and
recycling at least a portion of the liquid stream to step i. of the process for use as, or as part of, the absorption or dissolution medium,
wherein the pH of the absorption or dissolution medium is controlled to maintain the HCO₃⁻:CO₃²⁻ molar ratio in the reagent stream at least at about 0.8.

2. The process according to claim 1 comprising contacting the feedstock with the absorption or dissolution medium in a gas contactor or absorption column.

3. The process according to claim 2 further comprising concentrating the reagent stream in the gas contactor or absorption column.

4. The process according claim 2 or claim 3 wherein the liquid stream is recycled to the gas contactor or absorption column.

5. The process according to any one of claims 1 to 4 wherein the pH of the absorption or dissolution medium is at least partly controlled by adjusting the pH of the recycling liquid stream.

6. The process according to claim 5 wherein the recycling liquid stream is pH adjusted before supply to step i.

7. The process according to claim 5 or claim 6 wherein the recycling liquid stream is pH adjusted to increase its pH to from about 10.5 to about 11.75.

8. The process according to any one of claims 5 to 7 wherein the pH of the recycle stream is controlled by the addition of an alkaline aqueous make-up stream.

9. The process according to claim 8 wherein the alkaline aqueous make-up stream comprises sodium hydroxide.

10. The process according to claim 8 or claim 9 wherein the alkaline aqueous make-up stream has a pH of about 12 to about 13.

11. The process according to any one of claims 7 to 10 wherein the recycling liquid stream is pH adjusted to increase its pH to from about 11.0 to about 11.6 or to about 11.5.

12. The process according to any one of claims 1 to 11 wherein the reaction mixture in the bioreactor has a pH of greater than or equal to 10.

13. The process according to any one of claims 1 to 12, wherein the pH of the bioreactor reaction mixture in the bioreactor is at least partly controlled by the injection of a gas comprising carbon dioxide.

14. The process according to claim 13 wherein the carbon dioxide is sourced from waste or a by-product of a downstream process, optionally wherein the downstream process comprises a biofuel production process.

15. The process according to any one of claims 1 to 14 wherein the bioreactor reaction mixture comprises enzymes and/or a solvent selected from at least one omine, alkaline solution, wastewater, and/or ammonia.

16. The process according to any one of claims 1 to 15 wherein the bioreactor reaction mixture comprises biological means selected from at least one of photoautotrophs, non-photosynthetic, non-photoautotrophs, auto-chemo-lithotrophs, alkaliphiles, thermophiles, engineered microbes, bacterial consortia, enzymes and/or proteins.

17. The process according to any one of claims 1 to 16 wherein the microbial broth efficiency of carbon dioxide removal is at least 50%, or at least 60%, or at least 70%.

18. The process according to any one of claims 1 to 17 wherein the HCO₃⁻:CO₃²⁻ molar ratio in the reagent stream is at least about 0.9, and the pH of the absorption or dissolution medium is controlled to maintain the HCO₃⁻:CO₃²⁻ molar ratio in the reagent stream at least at about 0.9.

19. The process according to any one of claims 1 to 18 wherein the biomass-containing reaction product is at least partly, optionally wholly, inanimate.

20. The process according to any one of claims 1 to 19 wherein the microbial broth and is at least partly, optionally wholly, non-algal and/or non-photosynthetic.

21. An apparatus for sequestering carbon dioxide to produce a biomass containing reaction product, the apparatus comprising:
a bioreactor for producing a biomass containing reaction product from a fluid stream comprising CO₂;
a biomass separator configured to separate the biomass containing reaction product into biomass and a liquid stream;
means for monitoring and controlling the pH of the liquid stream; and
means for recycling at least part of the separated liquid stream to generate at least part of the fluid stream comprising CO₂.

22. The apparatus according to claim 21 further comprising a gas contactor or absorption column to concentrate the fluid stream.

23. The apparatus according to claim 21 or claim 22 configured to operate the process of any one of claims 1 to 20.

24. Biomass or biomass-containing reaction product produced by a process according to any one of claims 1 to 20.
